(19) United States
(12) Patent Application Publication
(10) Pub. No.: US 2026/0079819 A1
(43) Pub. Date: Mar. 19, 2026

(54) Test of Legacy Code for Device Control of Medical Technical Devices

(71) Applicant: Fresenius Medical Care Deutschland GmbH, Bad Homburg (DE)

(72) Inventor: Ingo Schäfer, Eschborn (DE)

(21) Appl. No.: 19/109,036

(22) PCT Filed: Sep. 11, 2023
(86) PCT No.: PCT/EP2023/074862

§ 371 Date: Mar. 5, 2025

(30) Foreign Application Priority Data
Sep. 13, 2022 (EP) 22195302.9

(51) Int. Cl. G06F 11/3668; G06F 11/3698; G16H 40/40

(52) U.S. Cl. CPC G06F 11/368; G06F 11/3696; G06F 11/3698; G16H 40/40

(57) ABSTRACT

The present disclosure relates to testing software code of a productive system for a medical technical device, such as a dialysis machine or a blood treatment device, when hardware-side and/or software-side changes are made to the productive system. The software code may be legacy code. When operating the productive system with the legacy code, field data is collected that is at least indirectly considered in the test.

### CROSS-REFERENCE TO RELATED APPLICATIONS

The present application is the national stage entry of International Patent Application No. PCT/EP2023/074862, filed on Sep. 11, 2023, and claims priority to Application No. EP22195302.9, filed on Sep. 13, 2022, the disclosures of which are incorporated herein by reference thereto.

### TECHNICAL FIELD

The disclosure relates to a functionality test of software code, e.g., legacy code, which is used to control and/or operate a productive system for a medical technical device, e.g., a dialysis device. The productive system comprises hardware on which the software code to be tested is installed. When changes are made to the productive system, the software code may be subjected to a functionality test, e.g., for specification-compliant functionality. The disclosure further relates to a method for functionality testing, to a test system, to a medical technical device, to a medical treatment system, and to a computer program.

### BACKGROUND

Today, software for controlling complex devices and systems. e.g., medical technical devices, is often based on outdated, monolithic program code (legacy code) that has been continuously developed over decades. The disadvantage here is that current programming standards and regulatory requirements change over time. Especially in the medical sector, the requirements in the area of software security are continuously increasing. One example of this is the requirement to provide up-to-date software tests, such as unit tests, in order to comply with approval and safety-relevant regulations so that the device software and the underlying medical technical device can continue to be operated.

In many cases, however, legacy code can only be subjected to modern software tests at great expense. Due to obsolete software and hardware structures, for example, it is generally only possible to a limited extent from a technical point of view to perform unit tests or to generate meaningful results from them. For example, alleged errors are determined on the basis of outdated legacy code segments, which turn out to be false-positive results upon closer analysis and examination. However, precise analyses of individual results are usually impossible with extensive program code that can be found, for example, in a large number of medical technology devices. However, due to positive field experiences with legacy systems, i.e., the combination of an original hardware implementation and a used system software based on legacy code, this problem is initially only critical to a limited extent. The reason for this is an empirical examination of so-called legacy systems (i.e., legacy software code implemented on a hardware) on the basis of a documented operating history spanning many years and free of errors. However, if, e.g., individual hardware components of the legacy system are modified, e.g., the main processor or the motherboard, a new regulatory review may become necessary. In fact, to ensure patient safety, this is usually unavoidable, since positive field experience can only be applied to the original combination of hardware implementation and system software, and a combination of new hardware implementation and outdated system software can lead to potentially unpredictable system behavior. A comparable situation arises analogously in the case of software changes while retaining an original hardware implementation. The limited applicability of modern software test procedures for quality assurance purposes in the case of legacy code or legacy systems consequently represents a problem that has so far only been inadequately solved from a technical point of view.

### SUMMARY

The methods, tests, systems, devices, and programs described in this disclosure can enable or improve verification or testing of the functionality of software code when changes are made to a computer-aided productive system. For example, the verification shall be possible even if the software code is legacy code.

The disclosure proposes a computer-implemented method, a computer program, a test system, a medical technical device comprising such a test system, and a medical treatment System.

In the following, a computer-implemented method is described. Features, advantages and/or alternative embodiments mentioned herein are likewise to be applied to the other devices, systems, and methods, and vice versa. In other words, the subject matter (directed, for example, to an apparatus, a system or a computer program) may also be further formed with the features described in connection with the method and vice versa. The corresponding functional features of the method are thereby formed by corresponding device modules, e.g., by hardware modules or microprocessor modules, of the system of product, and vice versa. The embodiments described above in connection with the method are not explicitly repeated for the device. In general, in computer science, a software implementation and a corresponding hardware implementation (e.g., as an embedded system) are equivalent. For example, a method step for “outputting” a test result may be performed with an output interface and the test results. Therefore, to avoid redundancy, the apparatus is not explicitly described again, although it may also be used in the alternative embodiments described with respect to the method.

According to a first aspect, the disclosure relates to a computer-implemented method for functionality testing of software code of a productive system. The productive system may be configured to control and/or operate a medical technical device. The productive system may be operated for the medical technical device. The functionality test of the software code is to be carried out, e.g., if the productive system is modified by changes to its hardware and/or to its software code, thereby creating a modified system. The modified system and the (previous) productive system may exist in parallel for a configurable time phase. The method may comprise the following:
Executing a test procedure on the productive system and generating an initial test result;
Executing the test procedure on the modified system and generate a second test result;
Comparing the first test result with the second test result to verify specification-compliant functionality of the modified system;
Outputting of a test result.

In a first variant, the test procedure can be executed directly on the productive system and/or the modified system. In a second variant, the test procedure includes the execution of a test suite on the productive system and/or the modified system.

The following is a more detailed explanation of the terms used in the application.

Comparing the first test result with the second test result provides the test result, which is then final and/or can be output.

The medical technical device is a technical device for medical purposes and/or medical treatments. It comprises medical units and computer-based or electronic units that essentially serve to control and/or operate the medical technical device. In an embodiment, the medical technical device is a blood treatment device, e.g., a dialysis device. The medical technical device may also be a peritoneal dialysis device or a plasmapheresis device. Dialysis is commonly used to replace kidney function by removing waste toxins and excess water. During treatment, and therefore during operation of the medical technical device, proper operation can be provided. Correct operation of the medical technical device requires correct functionality of the software code, which is implemented on the productive system and must be subject to verification.

The productive system is or comprises a computer-based system. The productive system can be operated on a medical technical device or operated for a medical technical device (e.g., as a server solution). Thus, the productive system may be formed at least in part directly on the medical technical device; alternatively or cumulatively, the productive system may be formed at least in part on a different computer system (than the productive system, e.g., in the form of a cloud-based solution) that is connected to the productive system and/or the medical technical device via an appropriate interface connection. The productive system may comprise an electronic unit. The electronic unit may comprise hardware, e.g., different hardware components, such as a CPU, a memory and/or physical interfaces, etc., and/or software code.

The electronic unit may comprise a computing system (e.g., in the form of an electronic circuit, such as an FPGA, or an embedded system, a system-on-chip, SOC, or in the form of an IC device) with any combination of hardware and software components. Examples may include a combination of a given hardware implementation (e.g., main computing unit, memory, and motherboard) that can be controlled based on software residing on volatile/non-volatile memory. For example, computing systems can be used as part of a treatment system (e.g., an infusion or dialysis system) to control individual components of the treatment system (sensors, pumps, etc.).

The test procedure for the computing system can in principle include all types of common software test procedures which are suitable for ensuring specification-compliant functionality between software and hardware implementation. Examples are unit tests, which test individual program sections (“units”) within a program code, e.g., functions or classes. Usually, so-called “mocks” are first created for this purpose, which allow individual program sections to run independently.

The actual program test is performed on the basis of the identified units, the generated mocks and the corresponding test inputs. To generate the test inputs, a “symbolic simulation” is used, whereby the unit to be tested is converted into a logical expression. Subsequently, the possible input value range of the unit to be tested is checked by controlling the behavior of the unit concerned. The program test thus makes it possible to identify precisely those units which, for example, can show an unforeseen behavior (e.g., a system crash of the computing system) due to an error.

The software code can be a so-called legacy code. It may be implemented as a monolithic block. Legacy software code is “old” code (not newly programmed and already in use in the field for a long time) that has been operated in the devices or productive systems for a long time and is operationally bound to a specific version of an operating system and/or hardware model. The legacy software may be written in a legacy programming language and may reside on mainframes, for example. The software code may be present and stored in any programming language. The software code may be machine code (e.g., in assembler).

A software code section is a part or a section of the software code. The software code section can be an extracted code module. The extraction is performed automatically using a decomposition algorithm according to configurable decomposition criteria.

The productive system and, e.g., its hardware and/or its software code is subject to changes (modifications). These changes result in a modified system. The modified system is also a computer-based system. The modified system is quasi based on the productive system and additionally includes the changes to the hardware and/or to the software code. The productive system and the modified system may exist in parallel for at least a configurable period of time, e.g., during which the functionality testing procedure is executed. It is usually necessary, for example, to replace obsolete hardware with newer hardware. Replacing the “old” hardware with the “new” hardware in combination with the “old” (i.e., original) software code results in a modified system. The modified system and, e.g., its software code must be tested, especially for specification-compliant functionality. Alternatively, the modified system can be created by keeping the “old” hardware (unchanged) but changing all or part of the software code deployed on the hardware.

The functionality check is a check of the software code or its sections, e.g., units. The software code or its sections are checked to see whether the provided functionality also corresponds to that which has been defined in a specification.

The test procedure is a part of the functionality test. The test procedure tests the software code sections, e.g., so-called units, for error-free functionality. The test procedure tests the software code sections, e.g., against a predetermined specification for the software. According to the disclosure, the test procedure can be applied repeatedly, e.g., twice on different computer-based systems. A first application of the test procedure tests or verifies the (runtime) behavior of the compiled software code section on the old, unmodified productive system, and a second application of the same test procedure tests or verifies the (runtime) behavior of the compiled software code section on the modified system. The test procedure may be intended to compare the behavior of the software code section with the TARGET behavior (given, e.g., by a specification) or against an expected behavior (e.g., according to the behavior on the unchanged productive system).

For the old system, e.g., the legacy system, there is usually a large amount of field data that characterizes error-free operation. This data can also be referred to in its entirety as “field experience”. For example, a positive field experience includes that the input data generated in the field results in output data that conforms to specifications. The field experience advantageously and/or e.g., indirectly enters into and/or can be taken into account in the functionality test. This is done by first executing the test procedure as a first test procedure on the productive system, which serves as a reference or benchmark system, as it were, and of which it is known that it has functioned faultlessly for some time in the past. The first test result thus represents error-free operation by definition. For example, the first test result represents error-free operation even if the first test result provides a set of errors (this may be the case, for example, if the errors are not relevant or are within a tolerance range for the medical technical device). In the test procedure, input data are fed as test data to the respective software code section and compared with the output or result data.

The productive system (with the legacy software code) has been in operation for a long time without errors, so field data representing error-free operation could be collected.

The field data may be sensor data acquired during productive system operation and which allow conclusions to be drawn about specification-compliant functionality of the respective computing system (productive system or modified system). The field data can be collected automatically via sensors. For example, this may include an error rate and/or error reports of a computing system within a certain period of time, e.g., within several years. A specification-compliant functionality of the computing system would be given, for example, if the error rate does not exceed certain regulatory limits and/or the transmitted error counts and/or error types, have relatively few errors, whereby these have only a low safety relevance, i.e., comply with regulatory requirements (e.g., IEC 61508 or IEC 62304 in medical technology). In this context, a specification-compliant functionality of the computing system could also be given if no safety-critical errors could be recorded in a defined period of time.

In some embodiments, the test method is or comprises a unit test. A unit test is a test or examination in the software development process in which the smallest testable parts or elements of an application, called units, are examined individually and independently of one another to determine whether they are operating properly or in accordance with specifications. For further details of the approach, which is well known in itself, please refer to the publication Hamill, Paul (2004). Unit Test Frameworks: Tools for High-Quality Software Development. O'Reilly Media, Inc. ISBN 9780596552817. Alternatively, the test procedure may be designed as a regression test, which is used to repeat test cases. The disclosure enables testing of a modified system in comparison to a legacy system.

In principle, the method or test system according to the disclosure can also be used at higher test levels (e.g. for integration tests, system tests). However, this is only advantageous if other aspects (e.g. synchronization) are thereby included in the test results, which are omitted at the more detailed levels.

In the simplest case, the test result can be a “pass/fail” and thus a binary result. Alternatively, it may include other metadata, such as information about a test coverage. It may also specify thresholds, e.g., a degree of agreement between the first and second test results. In a configuration phase, it can be configured whether the test is considered “successfully completed” or “passed” only if there is a 100% match between the first and second test results, or whether a lower match should also be considered successful, e.g., in a range of 80-100%, e.g., between 90% and 100%. A fuzziness (no 100% match) comes into question especially for pseudo-analog quantities (e.g. rounding differences for real numbers) or non-functional parameters (e.g. computing time requirements, memory requirements, response times, etc.).

In another embodiment, the method comprises at least one of the following:
Automatic decomposition of the software code into software code sections;
Automatically creating a test environment for one software code section at a time of all software code sections and/or
Automatic creation of test stimuli or input data for the execution of the test procedure. (Test results are created during or after execution of the test and also represent test data).

The specific creation of a test environment for one software code section at a time has the advantage that the test environment can be created in a targeted manner and/or tailored to the software code section and/or its functionality. Further a minimum test environment keeps the expenditure (e.g., computing time, memory place) with stimuli generation and/or test execution in limits. Usually very many tests are generated for each section.

A test environment is a computer-based environment or basis for the execution of the subsequent test. For example, the test environment contains parts that are prerequisites for the execution of the test object, but which are not part of the test object itself. Examples are global data or functions.

The test environment can be created automatically. In this application, “automatic” should be understood as “without user interaction”. Alternatively, the test environment can also be created semi-automatically through appropriate configurations or settings by the user.

In another embodiment, a decomposition of the software code into software code sections is performed automatically. For example, by applying formal user-selectable and/or configurable decomposition criteria.

The decomposition criteria may include file membership, naming, and/or syntactic and/or semantic properties of the software code and/or the software code section. For example, the “file membership” decomposition criterion checks the software code line by line to see if it belongs to the same file. As long as the evaluated lines belong to the same file, it is the same software code section; however, as soon as the line belongs to a different file, the end of the software code section is reached and a new software code section is generated. Likewise, other syntactic and/or semantic properties of the software code can be evaluated as decomposition criteria. These can be, for example, function boundaries, loop bodies, block boundaries. Alternatively or cumulatively, a preconfigurable number of lines of the software code can also be applied as a decomposition criterion.

Alternatively or in addition, decomposition criteria can be formed by formal criteria, i.e. criteria that are applied formulaically to arbitrary parts of an implementation under test, e.g. on the basis of file storage, syntactic elements, names, etc. For example, each function of a C program can become a section to be tested.

Alternatively or in addition, decomposition criteria can be freely specified. In this case, the decomposition is performed on the basis of manually defined limits, e.g. a concrete list of desired entry points and/or concretely named parts that are not to be cut off.

The decomposition criteria can be formed by formal criteria and can be freely predefined in order to be able to test even complex implementations efficiently.

One or more decomposition criteria can be applied. In a configuration phase, you can define which of the decomposition criteria and/or in which form (e.g. threshold values, limit values) they are to be applied.

For example, the creation of a test environment for one software code section of all software code sections is iterative, in that an initially automatically created test environment is successively automatically supplemented by missing elements.

The initial test environment may be empty or may have resulted from an analysis of some kind. Alternatively, you can start with a manually defined test environment. For example, the aforementioned definitions are determined depending on the language and implementation. For example, in C/C++ an empty test environment can be used. If the execution of the test environment fails, for example, due to a missing declaration of a function, such a function is automatically added and the executability is checked again. The behavior of the corresponding function (e.g., return values) can be controlled by additional, test-dependent input data, which is then also automatically generated in the following step. Accordingly, missing type definitions, missing global variables, etc. can be processed. Elements whose definitions are not obvious (for example data types) can be created according to the definition of the original software in the test environment. This is possible by means of syntactic analysis of the previous software.

Missing elements can be identified, for example, by diagnostic messages when trying to execute them. This is also language-dependent. In C/C++, for example, the error messages of the compiler and/or linker can be processed by the method according to the disclosure. The supplementing can be done in C/C++, for example, by adding corresponding objects or types with the corresponding data types or methods and signatures to the test environment. The necessary information can be extracted from error messages or the overall code of the implementation.

Since even with arbitrary decomposition of the software each software part can have only limited dependencies, an iterative creation of the test environment converges after finitely many steps.

The generation of test stimuli or input data can be performed by a so-called “symbolic execution” algorithm on one decomposed software code section at a time, including the generated test environment. For further details on symbolic execution, please refer to KLEE: “Unassisted and Automatic Generation of High-Coverage Tests for Complex Systems Programs”, Cristian Cadar, Daniel Dunbar, Dawson Engler, 8th USENIX Symposium on Operating Systems Design and Implementation, 2008. In symbolic execution, instead of concrete input values for the unit under test (software code section), abstract input data, e.g., a logical expression, e.g., in the form of a term and/or a function, is used and the respective software code section (e.g., the unit) is subjected to a path analysis. As “path” in this context an execution pad of the program is understood at run time, thus which program branches are pursued due to the input data. If a program is symbolically executed along a path, then to each point in this execution a value and a path condition can be stored associated.

The execution of a test procedure on the productive system and/or on the modified system including the created test environment can be performed for each disassembled software code section and/or for each set of test stimuli or input data created for it, e.g., separately and/or individually. Since these combinations test different aspects, they must be executed separately. Alternatively or additionally, the execution of the test procedure on the modified system including the created test environment can be performed for each decomposed software code section and/or for each set of test stimuli or input data created for it, e.g., separately and/or individually.

Parallelization (of the execution of the above-mentioned different combinations of test procedures) is possible and makes sense; however, the independence of the executions must also be ensured in this case. Test results and test executions must always be independent of tests executed previously and/or in parallel in order to exclude mutual influence.

Alternatively or in addition, the generated first and/or the generated second test result can be digitally processed and/or stored as a digital unique code, e.g., as a checksum. This has the advantage that less storage space is required and that, if necessary, the performance of the method can be increased, since the comparison of checksums can be performed more quickly than of more extensive test results. The use (processing and/or storage) of the digital unique code can be determined depending on the system under test. It is also possible to store the generated first and/or second test result in addition to the respective digital unique code, for example for analysis in case of deviations.

Outputting a test result may include outputting a test coverage. This can improve the reliability of the procedure.

Creating a test environment for one software code section at a time may include user interaction. For example, the creation of the test environment may comprise a complete or partial transfer of software code sections of the productive system (the unchanged or unmodified implementation) into the test environment. For example, if test environment behavior controlled by test data generation (stimuli generation) does not result in adequate test coverage (e.g., because stimuli generation procedures would otherwise require too much computing time/memory), it may be useful to compose the test environment at least partially from parts (e.g., individual functions) of the original implementation (e.g., legacy code).

It is also possible to replace individual, automated steps of the functionality testing procedure described above with manual user interactions, e.g., when creating the test stimuli or input data or stimuli or the test environment, or by manually adjusting the automatically obtained artifacts, e.g., supplementing the stimuli, to increase test coverage.

Alternatively or in addition, further actions can be initiated automatically based on the deviations found in the comparison between the first and second test results, such as creating manual tests, an additional review of critical details, and/or an adjustment of the modified implementation.

Alternatively or in addition, further measures can be triggered automatically in response to the achieved test coverage, such as the execution of additional manual tests in case of low test coverage and/or a refactoring of the software based on the achieved test coverage. “Refactoring” the software code in this context means revising the structure of the source code without changing anything in its functionality or observable behavior. The aim of refactoring is to structure the code better, to make it more readable, e.g., also to make it easier to maintain and/or to be able to develop it better and to make the software more performant.

Alternatively or in addition, the automatically generated software code decomposition into software code sections proposed here and created test environments can be used as the basis for refactorizations. In addition to dependencies between software parts, the degree of commonality between the respective test environments is a useful measure for refactorizations.

With the solution according to the disclosure it is possible that the field data or the totality of the field data in the form of field experience is at least indirectly included in the test. In this context, “indirectly” means that the field data is not necessarily required explicitly as an “argument” in the test. The field data is only implicitly included in the test, e.g., as a data record that represents that no errors were observed on a certain set of machines in a certain period of time or that the system ran without errors. Alternatively, in a further development of the disclosure, the field data can also be processed directly and/or explicitly during the test. For this purpose, stimuli and optionally results can be stored in the field as part of the execution for already identified software parts. These can be used to determine test cases and/or results on the productive system or for comparison with results of the modified system.

This also makes it possible to replace previous manual system tests with automatically generated software tests. This means that generated, automatically repeatable software tests can reduce the need for manual tests even without changing the implementation.

In another aspect, the disclosure relates to a computer program, the computer program being loadable into a memory unit of a computer and including program code portions for causing the computer to execute the method of functionality testing as described above when the computer program is executed in the computer.

In a further aspect, the disclosure relates to a test system, which can also be designed as a test device, and is intended for executing the method described above. The test system is used for functionality testing of software code of a productive system for a medical technical device. The test system can be used when a modified system has been created from the productive system by changes to its hardware and/or software. The productive system may be operated and/or controlled with hardware and software code. The modified system may be operated and/or executed with modified hardware and/or software code. The test system is formed with:
A first test unit that initiates execution of a test procedure on the productive system and that is designed to generate a first test result;
A second test unit that causes the test procedure to be executed on the modified system and that is designed to generate a second test result;
A computing unit designed to compare the first test result with the second test result to verify specification-compliant functionality of the modified system;
An output interface for outputting a test result.

There are different implementation variants for the test system. For example, the test system can take the form of a test device. Alternatively or in addition, the test system can be provided as a separate additional device that can be connected to the production device via suitable logical and/or physical interfaces as required (i.e. temporarily for testing). Alternatively or in addition, the test system can be installed directly on the productive device itself, e.g. the dialysis machine.

The first test unit may be installed directly on the productive system and cause and/or initiate and/or control the execution of the test procedure. The first test unit can alternatively or in addition be installed on a separate device (separate from the productive system) and only be in data connection with the productive system.

The second test unit may be installed directly on the modified system (changed hardware and/or changed software, e.g. new software release or patch) and cause and/or initiate and/or control the execution of the test procedure. The second test unit may alternatively or in addition be installed on a separate device (separate from the modified system) and only be in data communication with the modified system.

The computing unit can be “deployed” on the hardware on which the first and/or second test unit is also installed or can be installed on a separate hardware with corresponding data connection.

The output interface may be formed on the productive system and/or the modified system and/or another device, such as a mobile terminal, e.g. a cell phone or a laptop or the like. The test system may be formed as a distributed system.

In a further aspect, the disclosure relates to the use of the method and/or test system described above to control further measures. Further measures may relate, for example, to refactoring and/or manual test generation.

In another aspect, the disclosure relates to a medical technical device comprising a test system or units/parts thereof as described above.

In another aspect, the disclosure relates to a medical treatment system for medical treatment of patients, comprising an electronic unit, wherein functionality testing of functions of the electronic unit is performed by a software code functionality testing method as described above.

In the following detailed description of the figures, non-restrictive examples of embodiments with their features and further advantages are discussed on the basis of the drawing.

### BRIEF DESCRIPTION OF THE FIGURES

FIG. 1a shows a productive system P from which a modified system P′ is generated by at least one modification to software code installed on hardware, and a test method for execution on the productive system P and the modified system P′ to generate first and second test results;

FIG. 1b shows a productive system P from which a modified system P′ is generated by at least one modification to the hardware on which software code is installed, and a test method for execution on the productive system P and the modified system P′ to generate first and second test results;

FIG. 2 a flowchart of a process 200 according to an embodiment;

FIG. 3a shows an embodiment example in which the productive system P is installed on a medical technical device DG, and

FIG. 3b another embodiment in which the productive system P is operated on a separate computing unit for a medical technical device DG;

FIG. 4 a schematic overview diagram of a test system 400 with further components;

FIG. 5 an embodiment in which the test system 400 is implemented on the medical technical device DG, and

FIG. 6 another embodiment in which the test system 400 is a distributed system;

FIG. 7 shows a flowchart of a method 700 according to an embodiment; and

FIG. 8 shows a schematic overview of a test system 400 of FIG. 4 in another embodiment.

### DETAILED DESCRIPTION

The present disclosure relates to a computer-implemented method for functional verification or testing of so-called legacy software program code on, for example, modernized hardware implementations using field data.

Medical technical devices DG, such as dialysis machines or blood treatment devices, comprise a large number of mechanical, electronic and/or physical components. The electronic components may be combined in the form of an electronic unit. The electronic unit may be used to operate and/or control a productive system P. The electronic unit and/or the productive system P may comprise a hardware HW on which a software code SW is installed. During the lifetime of the medical technical device DG and/or the productive system P for the medical technical device DG, the productive system P is subject to changes. Changes can be made to the software code SW and/or to the hardware HW. Both scenarios lead to a modified system P′.

Since the productive system P is basically used for a medical technical device DG, any changes to the productive system P must be checked, especially whether they meet the regulatory requirements in medical technology.

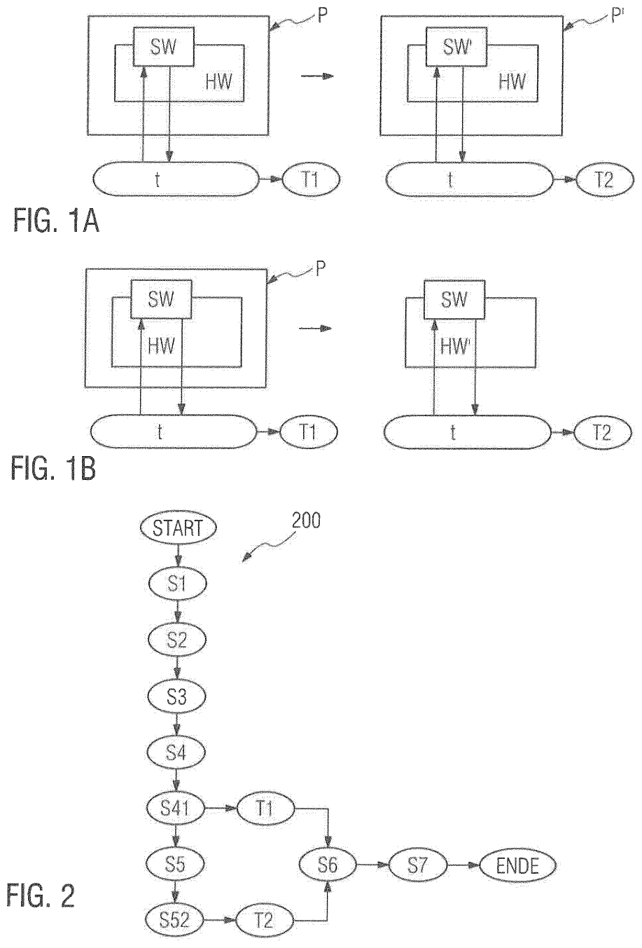

FIG. 1A shows an implementation example in which the changes are made to the software code SW->SW′ while the hardware HW remains unchanged. This can be caused, for example, by the application of a patch or a new software release.

FIG. 1B shows another embodiment example where the changes are not only made to a software code SW, but where a new hardware HW is used.

In both cases, a modified system P′ is created from the original productive system P. However, the productive system P and the modified system P′ can exist simultaneously. The modified productive system P′ must be tested in each case.

For this purpose, the software code is subjected to a test procedure t.

In the embodiment example of FIG. 1A, both the software code SW on the old unmodified productive system P is tested with the test procedure t (this is shown in FIG. 1A on the left side) and the modified software code SW of the modified productive system P′ (this is shown in FIG. 1A on the right side). The test procedure t executed on the productive system P generates a first test result T1 and the test procedure t executed on the modified system P′ generates a second test result T2.

In the example of FIG. 1B, the test procedure t is used to test both the software code SW on the old unmodified productive system P (this is shown on the left side in FIG. 1A) and the software code SW on the modified hardware HW of the productive system P′ (this is shown on the right side in FIG. 1A). The test procedure t executed on the productive system P generates a first test result T1 and the test procedure t executed on the modified system P′ generates a second test result T2.

In a later step of the functionality testing method, the first test result T1 can be compared with the second test result T2 for concordance (matching) to generate and/or output a (final) test result TE.

FIG. 7 describes a flowchart of a method 700 for functionality testing according to an embodiment example. After starting the method, in step S4 the test procedure t can be executed on the productive system P to generate the first test result T1 in step S41. Subsequently, in step S5, the test procedure t can be executed on the modified system P′ to generate the second test result T2 in step S51. In step S6, the first test result T1 can be compared with the second test result T2. The comparison can be performed by an algorithm. In step S7, the final test result TE can be output, for example on an output interface AS (shown in FIG. 4).

FIG. 2 shows a flowchart of a method 200 for functionality testing according to another embodiment. After starting the method, a decomposition of the software code SW into software code sections can be performed in step S1. This can be performed algorithmically and automatically. The automatic decomposition of the software code SW can be performed, for example, on the basis of formal, preconfigurable decomposition criteria which can be selected by the user and which are offered to him or her for selection on a user interface after they have been configured in a configuration phase. These decomposition criteria may concern, for example, a file affiliation, a naming convention, and/or syntactic and/or semantic properties of the software code SW and/or software code sections. For example, they may be function limits (boundaries), loop bodies, block boundaries. Cumulatively or alternatively, the decomposition can be performed according to the number of lines of the software code SW.

In a subsequent method step S2, a test environment can be created automatically for each of the decomposed software code sections from the set of all software code sections. The automatic creation of the test environment (also referred to as a so-called “mock”) can be executed interactively by checking the executability of the respective software code with the previous (unchanged/unmodified) test environment and, if necessary, supplementing the test environment with missing elements.

In a further subsequent method step S3, test stimuli or input data can be created automatically, which are used to execute the test procedure t. For each identified or decomposed software code section including test environment, one or more sets of stimuli or input data/test stimuli or input data can now be generated automatically by so-called “symbolic execution”. This generates test stimuli or input data for the software code section and, if necessary, for the test environment.

In the subsequent process step S4, the test procedure t is applied to the productive system with the test stimuli generated in the previous process step to generate a first test result T1.

Alternatively, the test procedure t can be applied on the productive system with the field data. In the above alternative, however, the generation of the stimuli by means of symbolic execution/simulation is omitted.

Since it is known that the productive system P runs without errors in the unmodified version, the first test result T1 represents by definition an error-free state of the productive system P. This error-free state can be used as a reference or benchmark and for comparison with the second test result T2. The second test result T2 is generated automatically by executing the same test procedure t on the modified system P′.

In step S6, the first test result T1 can then be compared with the second test result T2 for concordance (consistency), in order to output a final test result TE in step S7, which is thus implicitly based on the state represented in the acquired field data (namely the previous-possibly error-free-operation of the productive system).

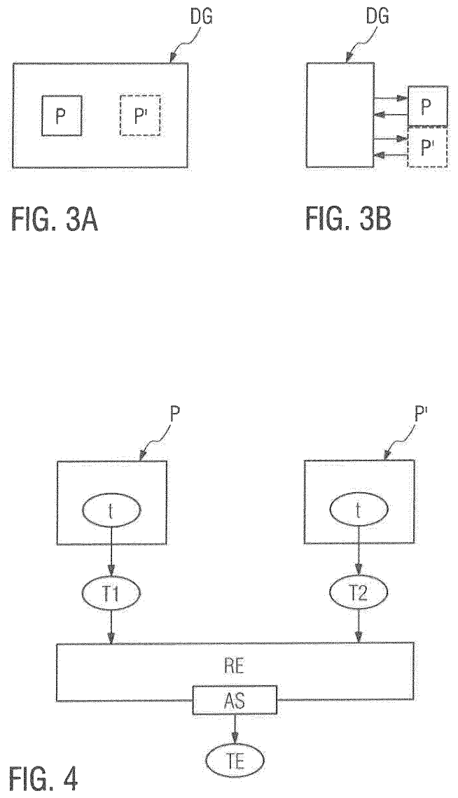

FIG. 3A shows an implementation option in which the productive system P and/or the modified system P′ is implemented directly and immediately on the medical technical device DG. Since the modified system P′ results from changes to the software code SW of the productive system and/or to the hardware HW of the productive system, the modified system is shown dashed.

FIG. 3B, on the other hand, shows an alternative implementation possibility in which the productive system P and/or the modified system P′ is/are not implemented directly on the medical technical device DG, but is/are operated for the medical technical device DG. The productive system P and/or the modified system P′ may be implemented on a separate computer or computer unit (e.g., programmable logic controller, SPL) that is in data communication with the medical technical device DG. As in FIG. 3A, the modified system is again shown dashed.

FIG. 4 shows a structural block diagram of a test system 400 with the productive system P on the left and the modified system P′ on the right. After execution of the test procedure t on the productive system P, the first test result T1 is automatically generated and after execution of the same test procedure t on the modified system P′, the second test result T2 is automatically generated. Thereupon, a comparison of the first test result T1 with the second test result T2 can be performed on a computing unit RE in order to output the (final) test result TE via the output interface AS. The test result TE is therefore a final test result.

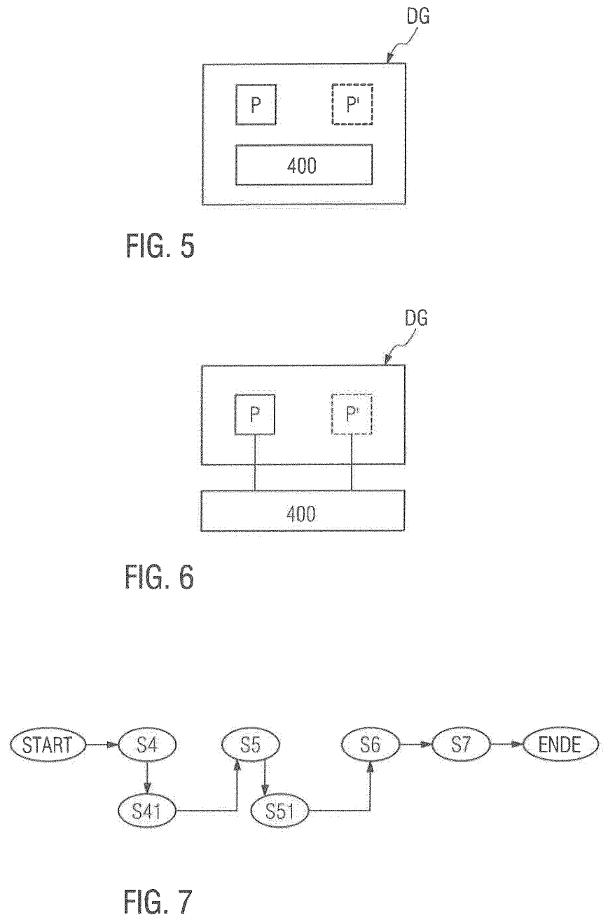

FIG. 5 shows an implementation example where the productive system P and/or the modified system P′ is/are implemented on the medical technical device DG and where the test system 400 is also implemented on the medical technical device DG.

In contrast, the implementation example of FIG. 6 shows that the productive system P and/or the modified system P′ may be implemented on the medical technical device DG, while the test system 400 is implemented as a distributed system on a separate computer that is in data communication with the productive system P, P′ of the medical technical device DG.

Finally, it should be noted that the description and the embodiments are in principle not to be understood restrictively with respect to any particular physical realization. All features explained and shown in connection with individual embodiments may be provided in different combinations in order to simultaneously realize their advantageous effects.

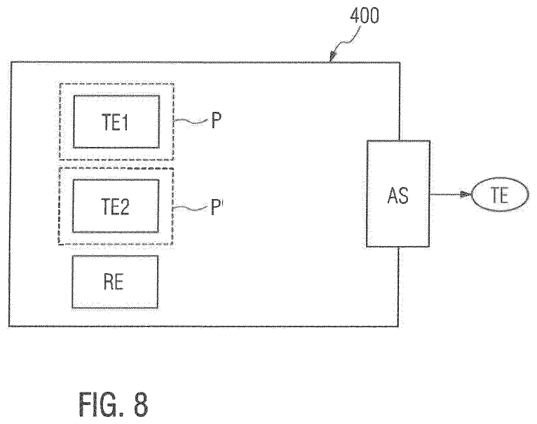

FIG. 8 shows a test system 400 with a first test unit TE1, a second test unit TE2 and a computing unit RE and an output interface AS for outputting a test result TE. The first test unit TE1 may be implemented on the productive system P (therefore shown dashed in FIG. 8, as this is only optional) and the second test unit TE2 may be implemented on the modified system P′ (therefore also shown dashed in FIG. 8, as this is only optional) to initiate the test procedure on the productive system P and on the modified system P′, respectively. The executions of the test procedures thus run on different systems. The test procedures can be executed simultaneously, in parallel or sequentially.

The scope of protection of the present invention is given by the claims and is not limited by the features explained in the description or shown in the figures.

The teachings in this disclosure can be applied not only to dialysis devices, but also to other medical technical devices (such as, for example, blood treatment devices or devices for the treatment of kidney diseases) or to non-medical technical devices comprising an electronic unit and/or operated and/or controlled by a productive system. Furthermore, the components of the test system 400 may be implemented distributed on multiple physical products.

### REFERENCE LIST

| | |
|---|---|
| DG | medical technical device, especially dialysis machine |
| P | Productive system |
| P′ | modified system |
| SW | Software code or software |
| HW | Hardware |
| SW′ | modified software code |
| HW′ | Modified hardware |
| RE | Calculation unit |
| AS | Output interface |
| t | Test procedure |
| T1 | first test result |
| T2 | second test result |
| TE | (final) test result |
| S1 | Decomposition |
| S2 | Creating a test environment |
| S3 | Creating test stimuli |
| S4 | Executing the test procedure on the productive system P |
| S41 | Generating a first test result for the test procedure |
| | executed in step S4 |
| S5 | Executing the test procedure on the modified system P′ |
| S51 | Generating a second test result for the test procedure |
| | executed in step S5 |
| 400 | Test system |
| TE1 | first test unit |
| TE2 | second test unit |
| 200 | Method for functionality testing of software code according |
| | to a first embodiment |
| 700 | Method for functionality testing of software code according |
| | to a second embodiment |

1-15. (canceled)

16. A computer-implemented method for testing functionality of software code of a productive system for a medical technical device when a modified system has been generated from the productive system by changes to its hardware and/or to its software code, the method comprising:
executing a test procedure on the productive system and generating a first test result;
executing the test procedure on the modified system and generating a second test result;
comparing the first test result with the second test result to verify a specification-compliant functionality of the modified system;
outputting a third test result.

17. The method of claim 16, wherein the test method is a unit test.

18. The method of claim 16, wherein the method comprises automatically performing decomposition of the software code into software code sections.

19. The method of claim 16, wherein the method comprises automatically creating a test environment for one software code section of each of the software code sections.

20. The method of claim 16, wherein the method comprises automatically creating test stimuli to execute the test procedure.

21. The method according to claim 16, further comprising automatically performing decomposition of the software code into software code sections by applying formal and/or user-configurable decomposition criteria.

22. The method according to claim 21, wherein the decomposition criteria includes a file affiliation, a naming and/or syntactic and/or semantic properties of the software code and/or the software code sections.

23. The method according to claim 16, wherein the method comprises performing decomposition of the software code into software code sections, and iteratively creating a respective test environment for a respective software code section of the software code sections, wherein an initially automatically created test environment is successively automatically supplemented by missing elements.

24. The method according to claim 23, wherein the method comprises creating test stimuli to execute the test procedure, and the creation of test stimuli is performed by a symbolic execution algorithm on a respective decomposed software code section including the created test environment.

25. The method according to claim 24, wherein executing the test procedure on the productive system and/or executing the test procedure on the modified system including the created test environment is performed for each decomposed software code section and/or for each set of test stimuli created.

26. The method according to claim 16, wherein the first test result and the second test result are processed and/or stored as a digital unique code.

27. The method of claim 26, wherein the digital unique code comprises a checksum.

28. The method according to claim 16, wherein the method comprises performing decomposition of the software code into software code sections, and iteratively creating a respective test environment for a respective software code section of the software code sections, wherein an initially automatically created test environment is successively automatically supplemented by missing elements, and the creation of a respective test environment for a respective software code section comprises a complete or partial transfer of software code sections of the productive system into the test environment.

29. The method according to claim 16, wherein outputting the third test result comprises outputting a test coverage.

30. A computer program, the computer program being loadable into a memory unit of a computer and including program code portions for causing the computer to execute the functionality testing method according to claim 16 when the computer program is executed in the computer.

31. A medical treatment system for medical treatment of patients comprising a medical technical device having a productive system comprising an electronic unit, wherein functionality testing of functions of the electronic unit is performed by the method of claim 16.

32. A test system for functionality testing of software code of a productive system for a medical technical device, if a modified system has been generated by changes to a hardware and/or to a software code of the productive system, wherein the test system is intended for executing a method, the test system comprising:
a first test unit for causing a test procedure to be executed on the productive system and which is intended to generate a first test result;
a second test unit for causing the test procedure to be executed on the modified system to generate a second test result;
a computing unit designed to compare the first test result with the second test result in order to check a specification-compliant functionality of the modified system;
an output interface for outputting a third test result.

32. A medical technical device comprising the test system according to claim 31.
